(19) United States
(12) Patent Application Publication
(10) Pub. No.: US 2008/0075775 A1
(43) Pub. Date: Mar. 27, 2008

(54) Tamsulosin controlled-release tablet

(75) Inventors: Yong Gan, Huairou (CN); Xinteng Zhou, Huairou (CN)

Correspondence Address: BEYER WEAVER LLP, P.O. BOX 70250, OAKLAND, CA, 94612-0250, US

(73) Assignee: Ocean Star International, Inc., Snowville, UT (US)

(21) Appl. No.: 11/580,215

(22) Filed: Oct. 11, 2006

(30) Foreign Application Priority Data
Sep. 22, 2006 (CN) 200610153091.8

(51) Int. Cl. A61K 9/24

(52) U.S. Cl. 424/473

(57) ABSTRACT

An osmotic pump device for delivery of tamsulosin or a pharmaceutically acceptable salt thereof to the gastrointestinal tract is provided. The device is a two-layer device comprising a drug containing layer containing as a carrier about 10 to 99 percent by weight of the first layer a polyvinylpyrrolidone polymer and/or copolymer, and a second layer comprising about 10 to 80 percent by weight of the second layer of water-insoluble volume-swellable polymers, about 80 to 10 percent by weight of the second layer of osmopolymers, and about 5 to 50 percent by weight of the second layer of osmagents.

### 1. RELATED APPLICATION

Priority is claimed pursuant to 35 USC 119(a) from Chinese patent application Serial No. 200610153091.8, filed Sep. 22, 2006, incorporated by reference herein in its entirety.

### 2. FIELD OF INVENTION

The present invention relates to a two-layer, osmotic pump device for controlled delivery of tamsulosin hydrochloride or a pharmaceutically acceptable salt thereof into the gastrointestinal tract.

### 3. BACKGROUND OF THE INVENTION

Tamsulosin hydrochloride is an alpha adrenoceptor-blocking agent developed by Yamanouchi Pharmaceutical Company and is used for the treatment of benign prostatic hyperplasia (BPH). It is common for the prostate gland to become enlarged as a man ages. Doctors call this condition benign prostatic hyperplasia (BPH), or benign prostatic hypertrophy. Many symptoms of BPH stem from obstruction of the urethra and gradual loss of bladder function, which results in incomplete emptying of the bladder. The symptoms of BPH vary, but the most common ones involve changes or problems with urination, such as: a hesitant, interrupted, weak stream; urgency and leaking or dribbling; more frequent urination, especially at night. Severe BPH can cause serious problems over time. Urine retention and strain on the bladder can lead to urinary tract infections, bladder or kidney damage, bladder stones, and incontinence or inability to control urination. Though the prostate continues to grow during most of a man's life, the enlargement doesn't usually cause problems until late in life. BPH rarely causes symptoms before age 40, but more than half of men in their sixties and as many as 90 percent in their seventies and eighties have some symptoms of BPH.

A common preparation of tamsulosin hydrochloride is a sustained-release capsule, where the primary mechanism of release is through dissolution and/or diffusion of drug from the capsule. However, the bioavailability of the tamsulosin by this device is greatly affected by presence of food in the digestive tract. Typically, the time to the maximum concentration (Tₘₐₓ) in the bloodstream is reached by 4 to 5 hours under fasting conditions and by 6 to 7 hours when the capsules are administered with food. Taking the sustained-release capsules under fasting conditions results in about a 30 percent increase in bioavailability and from about 40 to 70 percent increase in peak concentrations (Cₘₐₓ) compared to conditions when taken with food. Due to the effect of food on sustained-release capsules, patients taking these capsules under fasting conditions may experience the following side effects: dizziness, rhinitis and/or abnormal ejaculation. Therefore, it is desirable to develop a new preparation for delivery of tamsulosin hydrochloride which may avoid or alleviate the food effect of the sustained-release capsules, and which could also reduce the side effects when taken under fasting conditions.

Another kind of tablet containing tamsulosin hydrochloride is an oral controlled absorption system formulated as a hydrogel matrix. The mechanism of this tablet is to absorb water rapidly in the upper gastrointestinal tract to completely hydrate the gel material. This maintains a constant rate of releasing the drug and a consistent blood concentration of the drug for about 24 hours. However, this hydrogel matrix tablet is greatly affected by the intensity of gastrointestinal peristalsis, the process of wave-like muscle contractions of the alimentary tract that moves food along the tract. There can be large variations of peristalsis among individuals.

An osmotic pump device for controlled release of a drug is advantageous over the above-described sustained release capsules or tablets since it can be designed to have an excellent zero order release profile, is almost unaffected by pH of the environment, gastrointestinal peristalsis and food, and shows excellent in vivo and in vitro correlation. Delivery by an osmotic pump device typically avoids great fluctuations of blood concentration of the drug and reduces the frequency of administration, thereby increasing the probability of compliance by the patient.

An osmotic pump device for controlled release of tamsulosin hydrochloride is disclosed in US 2005/0100602 and US2005/0100603. In a disclosed device, the tamsulosin salt is contained in a layer with a hydrophilic polymer carrier. The particularly preferred carrier is poly(ethylene oxide), PEO. We find, however, that use of tamsulosin or a pharmaceutically acceptable salt of tamsulosin in an osmotic pump device using a polyvinylpyrrolidone (PVP) polymer and/or PVP copolymer as the carrier is surprisingly advantageous over PEO for several reasons. Firstly, PVP polymers and/or PVP copolymers as drug carriers, have a short lag time after administration to appearance of the desired physiological effect in the body. PEO can cause a rather extensive time lag in distributing the drug because of its slow speed of water absorption and hydration. In the environment of the gastrointestinal tract, the drug releasing properties of PVP polymer or copolymer carrier-containing osmotic pump devices are only rarely affected by the presence of food. Furthermore, PVP polymers and copolymers are more heat stable than PEO. The glass transition temperature of a linear PVP polymer such as Povidone ranges from 130° C. to 176° C., depending on the particular molecular weight. The T_{g} of Povidone (Plasdone K-90) is 174° C., and the T_{g} of Copovidone, copolymer of 1-vinyl-2-pyrrolidone and vinyl acetate (Plasdone S-630), is 105° C. The T_{g} of PEO polymers is typically in the range of 65° C. to 67° C. So PEO is not ideally heat stable and accordingly can be problematic both in the preparation of the osmotic pump device and during storage. For example, it is difficult to remove solvent during the granulation process required to form the drug-containing layer. Since the granulation temperature is not above 40° C., the residue of organic solvent would be high or it would take an abnormally long period of time to properly dry. During tablet pressing, the temperature will be increased because of friction. When the temperature is above 50° C., conglutinations may occur using PEO. Special equipment for cooling or slowing down compacting speed during tablet pressing becomes necessary. Similarly, the stored temperature of a PEO carrier must be relatively low in order to retain its ideal drug release characteristics. Thus, the storage of the devices will typically require careful temperature control. However, for tablets with PVP polymers and/or copolymers as the main carrier, no special cooling conditions or storage conditions are required.

These advantages of using PVP polymers and/or copolymers are added to the advantages inherent in an osmotic pump device compared, for example, to a hydrogel device in that variations of gastrointestinal peristalsis may be negated.

### 4. SUMMARY OF THE DISCLOSURE

The present invention is directed to osmotic pump devices for administration of the active ingredient tamsulosin or a pharmaceutically acceptable salt thereof at a controlled rate into the gastrointestinal tract comprising:

a) a core comprising a first layer containing a pharmaceutically effective amount of tamsulosin or a pharmaceutically acceptable salt thereof and about 10 to 99 percent by weight of the first layer of PVP polymer and/or PVP copolymer and a second layer comprising about 10 to 80 percent by weight of the second layer of water-insoluble volume-swellable polymers, about 80 to 10 percent by weight of the second layer of water-soluble osmopolymers, and about 5 to 50 percent by weight of the second layer of osmagents;

b) a wall surrounding the core comprising a semi-permeable material permeable to the passage of an exterior fluid present in the gastrointestinal tract and substantially impermeable to the passing of tamsulosin or the pharmaceutically acceptable salt thereof; and

c) a passageway in the wall communicating with the first layer and the exterior of the device for delivering the tamsulosin or the pharmaceutically acceptable salt thereof from the device.

Useful PVP polymers and copolymers as carriers in the first layer have an average molecular weight in the range of about 5000 to 3,000,000.

The second layer may comprise the sodium starch glycolate, low-substituted hydroxypropyl-cellulose (L-HPC), crosslinked carboxymethyl cellulose sodium or mixtures of two or more thereof as the water-insoluble, volume-swellable polymers. The second layer may further comprise water-soluble osmopolymers that act as drug-sustained release adjustors, selected from the group consisting of acrylic acid polymers, acrylic acid copolymers, such as Carbomers, hydroxypropyl-methyl-cellulose (HPMC), PVP polymers, such as Povidone, PVP copolymers, such as Copovidone, and mixtures of two or more thereof. This layer may further comprise an osmagent selected from the group consisting of a water soluble inorganic salt, mannitol, glucose, lactose, sucrose and mixtures of two or more thereof. A typical osmagent is sodium chloride.

The first and second layers may further comprise a glidant, a lubricant and a colorant. The lubricant may be selected from the group consisting of magnesium stearic acid, silicon dioxide and mixtures of two or more thereof.

This device may further comprise light blockers, plasticizers, pore formers, solvents, fillers, and a film coat for moisture resistance and color identification.

The passageway of the device typically has a diameter of about 0.2 to 1.2 mm.

The wall of the device typically comprises cellulose acetate or ethyl cellulose. The wall may also contain plasticizers.

### 5. BRIEF DESCRIPTION OF THE DRAWINGS

FIG. 1 shows the drug release curves from six samples of tamsulosin hydrochloride as described in EXAMPLE 3.

FIG. 2 shows the drug release of tamsulosin hydrochloride from Sample 1 as described in EXAMPLE 3.

### 6. DESCRIPTION OF THE PREFERRED EMBODIMENTS

The present invention will be described in terms of a two-layer osmotic pump device, typically in the form of a tablet. The term “layer” is used for ease of description, but it is understood that a layer may be a compartment containing the described materials. One of the layers is the drug containing layer or drug layer containing the drug tamsulosin or a pharmaceutically acceptable salt thereof and a carrier as well as other ingredients as described herein. The other layer is the push layer which contains various kinds of osmopolymers, which are water-soluble hydrophilic polymers that, when dissolved, produce osmotic pressure in the drug layer. The push layer also contains water-insoluble volume-swellable polymers, which have high water absorption speed and high water absorption capacity so that they swell intensively when exposed to water. The swell of these water-insoluble polymers can have a mechanical push force on the drug release layer. Either or both of the drug layer and the push layer may contain an osmagent, also called an osmotic solute. Such agents are typically salts such as sodium chloride, potassium chloride, and the like or compounds such as mannitol, glucose, sucrose, or other inorganic salts, organic salts or carbohydrates.

The device will be surrounded by a wall comprising a semi-permeable material. This material is permeable to the passage of an exterior fluid present in the gastrointestinal tract such as aqueous fluids and other biological fluids, and substantially impermeable to the passage of tamsulosin or a pharmaceutically acceptable salt thereof. Such semi-permeable materials include, but are not limited to, acetate cellulose, ethyl cellulose, cellulose diacetate, cellulose triacetate, and the like.

There will be at least one passageway in the device, particularly in the form of a tablet, in the wall communicating with the layer containing the active ingredient and the exterior of the device in order to deliver the active ingredient from the device. Typically this is an orifice provided by drilling the tablet with a laser from the exterior to the first layer containing the active ingredient. The size of the orifice will in part determine the drug (active ingredient) release rate. The diameter of such an orifice in a typical device ranges from about 0.2 to 1.2 millimeters.

The present invention provides a two-layer or two-compartment osmotic pump device, preferably in the form of a tablet for delivering tamsulosin or a pharmaceutically acceptable salt thereof, preferably a hydrochloride, to the gastrointestinal tract for absorption into the body. In general, if tamsulosin hydrochloride is the active ingredient, the tablet will contain up to about 2 percent by weight of tamsulosin hydrochloride based on the total weight of the tablet. In the drug containing layer, the tablet will advantageously contain as a carrier for the tamsulosin one or more PVP polymers and/or PVP copolymers, which comprise from about 10 to 99 percent by weight of the drug containing layer. The exact ratio of PVP polymer and/or PVP copolymer used within the drug layer is ultimately dependent upon the constituent of the drug core and the desired drug release character. A useful PVP polymer is Povidone, a synthetic homopolymer of linear 1-vinyl-2-pyrrolidone groups with a molecular weight in the range of about 5,000 to 3,000,000, typically about 1,300,000. A useful PVP copolymer is Copovidone, a copolymer of 1-vinyl-2-pyrrolidone and vinyl acetate in the mass proportion of 7:3, 3:2, 5:5 and 3:7, typically about 3:2. A typical molecular weight of Copovidone is about 50,000. Another useful PVP polymer is Crospovidone, a cross-linked 1-vinyl-2-pyrrolidone homopolymer.

The drug-containing layer also typically contains a glidant, such as silicon dioxide, a lubricant such as magnesium stearic acid and a colorant, such as an inorganic colorant to distinguish it from the push layer.

The drug-containing layer may also contain other ingredients such as hydrophilic sustained-release materials, dilutors, binders, and solvent. These additional sustained-release materials may be, for example, water-soluble osmopolymers such as acrylic acid polymers or copolymers, such as Carbomers, hydroxypropylmethyl cellulose (HPMC), and the like, or mixtures of two or more thereof. The drug-containing layer may also contain osmagents such as sodium chloride, lactose, mannitol, glucose, sucrose, fructose or mixtures of two or more thereof.

The second (push) layer of the device contains from about 10 to 80 percent by weight of the push layer of water-soluble osmopolymers, which can adjust the release ratio of the drug from the device, such as acrylic acid polymers or copolymers, such as Carbomers, hydroxypropylmethyl cellulose (HPMC), and/or PVP polymers and/or copolymers such as Povidone and Copovidone. The push layer also contains water-insoluble volume-swellable polymers, such as sodium starch glycolate, low-substituted hydroxypropyl cellulose, crosslinked carboxymethyl cellulose sodium, such as Croscarmellose sodium, and the like, or a mixture of two or more thereof. The push layer may also contain an osmagent such as sodium chloride, although the osmagent may also be contained in the drug-containing layer.

The push layer may also contain a glidant, such as silicon dioxide, a lubricant such as magnesium stearic acid and a colorant to distinguish it from the drug layer.

The colorants for the drug layer and push layer may be any one of several members of colorants, including inorganic oxides such as red ferric oxide, yellow ferric oxide, purple ferric oxide, black ferric oxide, or mixtures thereof.

Plasticizers may be typical plasticizers such as diethyl phthalate, ethyl phthalate, triethyl citrate, polyethylene glycol (PEG), or mixtures thereof.

Light blockers may be materials such as titanium dioxide, talc, silicon dioxide, or mixtures thereof.

Pore formers may be materials such as glycerin, propylene glycol, polyethylene glycol, water soluble inorganic salts, or mixtures thereof.

Solvents used in the manufacture of the drug layers may be materials such as acetone, water, ethanol, methylene dichloride, methanol, isopropanol, or mixtures thereof.

The membrane used to surround the device is a semipermeable composition such as cellulose acetate, cellulose diacetate, cellulose triacetate, or a cellulose alkanylate such as ethyl cellulose.

Methods of making osmotic pump devices are known in the art such as described by Santus et al., J of Controlled Release, 35, 1-21 (1995); and U.S. Pat. No. 4,765,989, the contents of which are incorporated by reference into the present specification.

A preferred method for making an osmotic pump device according to the invention is as follows. The drug layer is prepared by separately sieving a colorant, such as yellow ferric oxide, the PVP polymer and/or copolymer carrier, and other osmopolymer and/or osmagent to be used in the drug layer, through a 60-mesh sieve and homogeneously mixing the ingredients together with silicon dioxide which is used as a glidant. Then an alcohol water solution containing tamsulosin hydrochloride is sprayed into the dry mix, and the mixture powder is concomitantly granulated. Then magnesium stearate is added and mixed.

The push layer is formed by first homogenizing each ingredient by separately sieving through a 60-mesh sieve. Then the water-soluble osmopolymers, water-insoluble volume-swellable polymers, binders, osmagents and colorant are homogeneously mixed together with silicon dioxide as a glidant. An alcohol water solution is sprayed into the mixture to granulate it. Then magnesium stearate is added and mixed.

One layer is first compacted into the desired form and then the other layer is added and then the two layers are compacted together to form a two layer tablet. The semi-permeable membrane is coated on the outside of the tablet core and dried typically for about 24 hours at 45° C. A hole with the desired diameter, typically around 0.9 millimeters, is drilled into the wall adjacent to the drug layer by a machine or laser. The drilled tablet is coated with a moisture-proof film coat and dried again typically for about 12 hours at 45° C. This film coat can also improve the product appearance and offer color identification.

The controlled release tablet, according to the present invention typically maintains a relatively constant drug blood concentration for about 24 hours after administration. Advantageously, the release is not substantially affected by the environmental medium. That is, there is little individual variation in blood concentration due to variation in gastrointestinal peristalsis, pH, and food, with few side effects.

The daily dosage of tamsulosin or a pharmaceutically acceptable salt thereof may be determined on a case-by-case basis, but there is generally no particular limitation as long it is pharmaceutically effective for therapy or prevention of a particular disease or condition. For treatment of urinary dysfunction associated with BPH, typical daily dosages are in the range of about 0.1 mg 0.8 mg/day. Hence, tablets containing such amounts designed to deliver their payload of drug within 24 hours by sustained release would suffice.

The following examples are illustrative of the present invention, but they should not be considered as limiting the scope of the invention in any way.

### EXAMPLE 1

### Preparation of Simulated GI Media

Four kinds of dissolution medium simulating the gastrointestinal tract environment are designed to evaluate the effect of gastrointestinal peristalsis, pH and food on the drug releasing property.

### Medium A (SGF, simulated gastric fluid without pepsin):pH 1.2

| HCl | 7.0 | mL |
|---|---|---|
| NaCl | 2.0 | g |
| Water | sufficient | |
| Total volume | 1000 | mL |

### Medium B (SIF, simulated intestinal fluid without pancreatin):pH 6.8

| KH₂PO₄ | 6.8 | g |
|---|---|---|
| NaOH | 0.944 | g |
| Water | sufficient | |
| Total volume | 1000 | mL |

### Medium C (FaSSIF, simulated intestinal fluid, fasted condition):pH 6.8

| KH₂PO₄ | 3.94 | g |
|---|---|---|
| NaOH | adjust to a pH of 6.8 | |
| Sodium Cholyltaurine | 5 | mM |
| Lecithin | 1.5 | mM |
| KCl | 16.4 | g |
| Distilled water | sufficient | |
| Total volume | 1000 | mL |

### Medium D (FeSSIF, simulated intestinal fluid, fed condition):pH 5

| KH₂PO₄ | 8.65 | g |
|---|---|---|
| NaOH | adjust to a pH of 5 | |
| Sodium Cholyltaurine | 15 | mM |
| Lecithin | 3.7 | mM |
| KCl | 15.2 | g |
| Distilled water | sufficient | |
| Total volume | 1000 | mL |

Medium A represents standard condition of gastric fluid, Medium B represents the standard condition of intestinal fluid; Medium C represents the simulated condition of intestinal fluid (fasted condition); Medium D represents the simulated condition of intestinal fluid (fed condition). In use, administration under fasted conditions means administration without ingestion of food for at least 8 hours. Administration under fed conditions means that food is ingested within 30 minutes before or after administration.

The tablet releases drug slowly in the four selected media simulating the environment in vivo. The releasing properties are not affected by the media. This means that there is no statistically significant difference to restrict the clinical use. This provides a consistent and long-lasting curative effect with little individual variation and few side effects. Thus, the invention provides advantages of reduction of administration frequency, convenience for administration, little fluctuation of blood concentration, a long-lasting and steady curative effect, and few side effects.

### EXAMPLE 2

### Preparation of Tablets

These are several samples illustrating the invention.

| Prescription | Sample 1 | | Sample 2 | | Sample 3 | | Sample 4 | | Sample 5 | | Sample 6 | |
|---|---|---|---|---|---|---|---|---|---|---|---|---|
| Drug Layer | | | | | | | | | | | | |
| Tamsulosin | 0.2 | mg | 0.2 | mg | 0.8 | mg | 0.4 | mg | 0.2 | mg | 0.2 | mg |
| Hydrochloride | | | | | | | | | | | | |
| Copovidone | 129 | mg | 100 | mg | 114 | mg | 114 | mg | 89 | mg | 80 | mg |
| (Plasdone S630) | | | | | | | | | | | | |
| Povidone | — | | — | | — | | — | | — | | 34 | mg |
| (Plasdone K90) | | | | | | | | | | | | |
| Sodium Chloride | — | | — | | — | | — | | 40 | mg | — | |
| Yellow ferric oxide | 0.07 | mg | 0.07 | mg | 0.07 | mg | 0.07 | mg | 0.07 | mg | 0.07 | mg |
| Magnesium stearate | 1 | mg | 1 | mg | 1 | mg | 1 | mg | 1 | mg | 1 | mg |
| Silicon dioxide | 0.5 | mg | 0.5 | mg | 0.5 | mg | 0.5 | mg | 0.5 | mg | 0.5 | mg |
| Total amount of | 131 | mg | 102 | mg | 116 | mg | 116 | mg | 131 | mg | 116 | mg |
| drug layer | | | | | | | | | | | | |
| Push Layer | | | | | | | | | | | | |
| Sodium starch | 32 | mg | 32 | mg | 32 | mg | 35 | mg | 32 | mg | 35 | mg |
| glycolate | | | | | | | | | | | | |
| Hydroxypropyl | 14 | mg | 14 | mg | 14 | mg | 15 | mg | 14 | mg | 15 | mg |
| Methyl Cellulose | | | | | | | | | | | | |
| Carbomer | 5.7 | mg | 5.7 | mg | 5.7 | mg | 6.3 | mg | 5.7 | mg | 6.3 | mg |
| Sodium Chloride | 24 | mg | 24 | mg | 24 | mg | 26 | mg | 24 | m | 26 | mg |
| Copovidone | 18 | mg | 18 | mg | 18 | mg | 20 | mg | 18 | mg | 20 | mg |
| (Plasdone S630) | | | | | | | | | | | | |
| Red ferric oxide | 1 | mg | 1 | mg | 1 | mg | 1 | mg | 1 | mg | 1 | mg |
| Magnesium stearate | 0.5 | mg | 0.5 | mg | 0.5 | mg | 0.5 | mg | 0.5 | mg | 0.5 | mg |
| Silicon dioxide | 0.4 | mg | 0.4 | mg | 0.4 | mg | 0.4 | mg | 0.4 | mg | 0.4 | mg |
| Total amount of | 95 | mg | 95 | mg | 95 | mg | 104 | mg | 95 | mg | 104 | mg |
| push layer | | | | | | | | | | | | |
| Semipermeable | | | | | | | | | | | | |
| Membrane | | | | | | | | | | | | |
| Cellulose acetate | 33 | mg | 20 | mg | 18 | mg | 24 | mg | 22 | mg | 19 | mg |
| Total amount of | 259 | mg | 217 | mg | 229 | mg | 244 | mg | 248 | mg | 239 | mg |
| tablet | | | | | | | | | | | | |
| film coat | | | | | | | | | | | | |
| Spectrablend ™ | 21 | mg | 17 | mg | 18 | mg | 19 | mg | 20 | mg | 18 | mg |
| (CM 0165) | | | | | | | | | | | | |

CM 0165 is manufactured by Sensient Pharmaceutical Technologies. It comprises HPMC (hydroxypropylmethylcellulose), PEG, TiO₂ and pigment.

### Preparation Method

### 1. Preparation of the Drug-Layer Granules:

Yellow ferric oxide, and Povidone (Plasdone K-90) and/or Copovidone (Plasdone S630) and/or sodium chloride are sieved through a sieve of 60 mesh and homogeneously mixed together with silicon dioxide. Then the solid mixture is added into a fluid bed granulator, and a pre-prepared alcohol solution containing tamsulosin hydrochloride is sprayed to affect granulation. Water residue percent, drug content percent, content uniformity and related compounds are determined. Then magnesium stearate is added and mixed. Granules of drug layer are formed.

### 2. Preparation of the Push-Layer Granules:

First, all the ingredients are sieved through a sieve of 60 mesh separately, then Sodium CMS, HPMC, Carbomer, NaCl, Copovidone (Plasdone S630), red ferric oxide are mixed together with silicon dioxide. The mixture is added into a fluid-bed granulator, then 95% alcohol water solution is sprayed to make granulation. Water residue percent is determined. Then magnesium stearate is added and mixed. Granules of push layer is formed.

### 3. Tablet Pressing:

The two-layer tablet cores are compacted with the two types of granules. The diameter is 8 mm. Drug content percent and content uniformity of the two-layer tablets are determined.

### 4. Tablet Coating with Semi-Permeable Membrane:

The semi-permeable membrane is coated outside of the tablet core then dried at 45° C. for about 24 hours. The weight gain of tablet is controlled carefully during the coating process. The organic solvent residue amount is determined.

5. A pore with diameter of 0.9 mm is drilled in the wall adjacent to the drug-layer by machine or laser. The drug release profile is measured.

### 6. Tablet Coating with Moisture-Proof Film Coat:

The tablet after drilling is coated by the moisture-proof film coat then dried at 45° C. for about 12 hours. Then quality analysis is conducted fully, including the drug content percent, drug content uniformity, drug release profiles, related compounds, residue amount of acetone, and others.

### EXAMPLE 3

### Determination of Drug Release

The drug release profile is measured in a type 2 dissolution apparatus (paddle) according to the China Pharmacopeia at 37° C. in 500 ml water at 50 rotations per minute. At 3, 5, 7, 12, and 16 hours, 10-ml are sampled respectively and the same volume of new dissolution media is immediately added. The samples are filtered with 0.45-μm micropore filter membrane as test solution. A portion of tamsulosin hydrochloride reference substance is dried at 105° C. for 2 hours, dissolved in water-acetonitrile solution (65:35) and diluted to obtain a solution having a known concentration of 0.5 mg per mL. A portion of this solution is diluted with water to obtain a solution having a known concentration of 0.4 μg/mL as reference solution. Then 80 μl of test and reference solutions are respectively injected into the chromatograph to determine the release of drug at the different times for each tablet. The detection chromatograph system is as follows: the column is ODS packed, the mobile phase is acetonitrile-perchloric acid solution (Dissolve 8.7 ml perchloric acid and 3 g NaOH into 1900 mL water, adjust it with NaOH solution to the value of pH equal to 2.0, then add water to the full scale of 2000 ml) (35:65, v/v). The flow rate is 1.0 ml/min. The wavelength of determination is 225 nm.

### Results of Drug Release

TABLE 1
The average cumulative drug release percent
from six samples of tamsulosin hydrochloride

| Release | Sample | Sample | Sample | Sample | Sample | |
|---|---|---|---|---|---|---|
| percent/Time | 1 | 2 | 3 | 4 | 5 | Sample 6 |
| 1 h | 0 | 10 | 13 | 6 | 9 | 12 |
| 3 h | 16 | 33 | 40 | 28 | 29 | 34 |
| 5 h | 32 | 53 | 62 | 50 | 49 | 50 |
| 7 h | 47 | 76 | 80 | 70 | 68 | 67 |
| 12 h | 81 | 93 | 95 | 93 | 90 | 90 |
| 16 h | 93 | 96 | 96 | 96 | 92 | 95 |

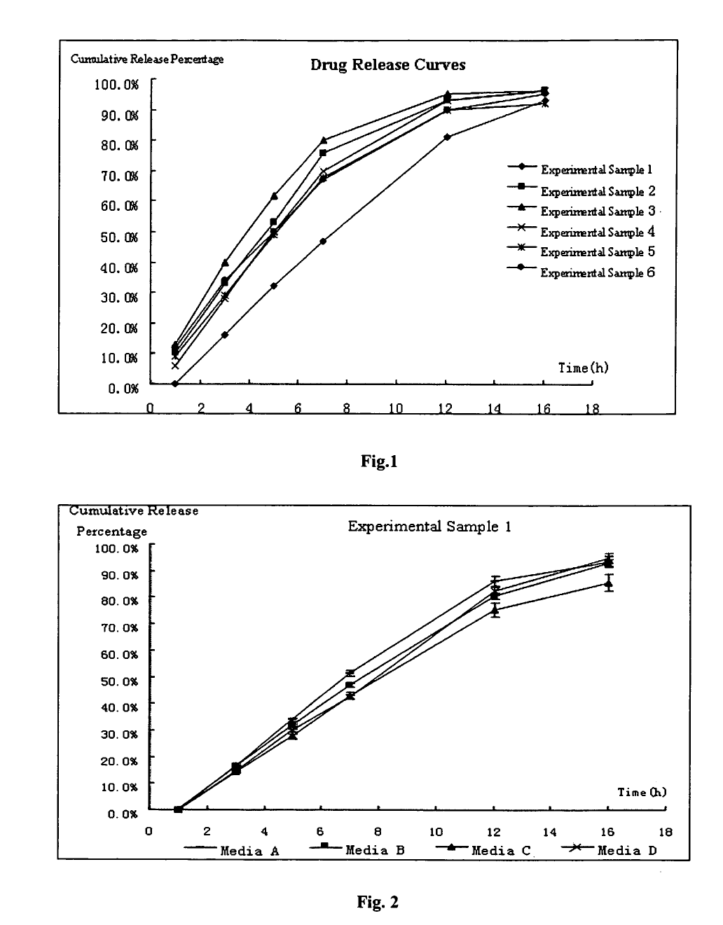

### The release curve is shown in FIG. 1.

Using sample 1, the gastrointestinal tract environment in vivo is simulated with the four media above-mentioned and the effects of food, pH and gastrointestinal peristalsis on the drug release are evaluated. The results of release are shown in FIG. 2.

### EXAMPLE 4

### Formulation of Tablet

| | | | Weight per 3000 tablets |
|---|---|---|---|
| Drug-Layer | 0.6 | g | Tamsulosin hydrochloride |
| | 387 | g | Copovidone(Plasdone S630) |
| | 3.0 | g | Magnesium Stearic acid |
| | 1.5 | g | Silicon dioxide |
| | 0.2 | g | Yellow Ferric Oxide |
| | 1000 | ml | Alcohol-water solution |
| Push-Layer | 96 | g | Sodium starch glycolate |
| | 42 | g | HPMC(K 15M) |
| | 17.1 | g | Carbomer (Carbopol 971P NF) |
| | 72 | g | Sodium chloride |
| | 54 | g | Copovidone (Plasdone S630) |
| | 3.0 | g | Red Ferric Oxide |
| | 1.4 | g | Magnesium Stearic acid |
| | 1.2 | g | Silicon dioxide |
| | sufficient | | Alcohol-water (95%) |
| | | | Amount used per 1000 tablets |
| Semipermeable membrane: | 50 | g | Acetate cellulose |
| | 1500 | ml | Acetone |
| Moisture proof film coat | 180 | g | Spectrablend ™ (CM 0165) |
| | | | powder |
| | 1000 | ml | water |

Preparation of granules in the drug-layer: First, 0.6 g tamsulosin hydrochloride is dissolved in 500 mL 60% alcohol-water solution. Then yellow ferric oxide, Copovidone (Plasdone S630) and silicon dioxide are mixed homogeneously after being sieved through a sieve of 60 mesh. Then the solid mixture is added into the fluid bed granulator, and the pre-prepared 500 mL alcohol solution containing tamsulosin hydrochloride is sprayed to affect granulation. The bed temperature is raised gradually to dry the granulation. Then the drug content percent, drug content uniformity and the residue water percent are measured. Then, magnesium stearic acid is added, and mixed. Granules of the drug-layer are formed.

Preparation of granules in the push-layer: First, all the ingredients are sieved through a sieve of 60 mesh separately, then Sodium CMS, HPMC, Carbomer (Carbopol 971PNF), NaCl, Copovidone (Plasdone S630), red ferric oxide and silicon dioxide are mixed homogeneously. The mixture is added to the fluid-bed granulator, and 95% alcohol water solution is sprayed to make granules. The bed temperature is raised gradually to dry the granules. The residue water percent is determined, and then magnesium stearic acid is added, and mixed. Granules of the push-layer are formed.

Tablet pressing: The two-layer tablet cores are compacted with the two types of granules. The diameter is 8 mm. The hardness should be above 8 kilograms. Drug content percent and content uniformity of the two-layer tablets are determined. The weight range of the drug layer is 127-135 mg, and the whole tablet core weight range is 222-230 mg.

Tablet coating: The semi-permeable membrane is coated on the outside of the tablet core. The weight of the semi-permeable membrane is 33.2 mg per tablet. Then it is dried at 45° C. for about 24 h. The acetone residue amount is determined.

A pore with diameter of 0.9 mm is drilled in the wall adjacent to the drug-layer. The drug release profile is detected, and then it is coated with the aqueous-based film coating solution and dried at 45° C. for more than 3 hours. Then quality analysis is performed, including the drug content percent, drug content uniformity, drug release profiles, related compounds, residue amount of acetone, and others. Then it is packed to form the final product.

### Tab. 2. Data of the cumulative drug release from tamsulosin controlled-release tablet in 500 mL water (method is as above mentioned, n=6)

| Time (h) | The average cumulative drug release percent |
|---|---|
| 3 | 16.2% |
| 5 | 33.8% |
| 7 | 49.7% |
| 12 | 84.3% |
| 14 | 90.2% |

### EXAMPLE 5

### Pharmacokinetic Data in Dogs

Six male beagle dogs, randomized to 3 groups, according to a Latin Square Design, 2 dogs per group, participated in a single-dose, 3-period, 3-crossover study.

In first period, the 2 beagles of group A took the reference drug (0.4 mg/2 capsules per dog) orally under fasting condition; the 2 beagles of group B took the test drug (0.4 mg/2 tablets per dog) orally under fasting condition; the 2 beagles of group C took the test drug (0.4 mg/2 tablets per dog) orally under fed condition.

After a 1-week washout period, the second cycle began. In second period, the 2 beagles of group A took the test drug orally under fasting condition; the 2 beagles of group B took the test drug orally under fed condition; the 2 beagles of group C took the reference drug orally under fasting condition. Then, there was a 1-week washout period.

In third period, the 2 beagles of group A took the test drug orally under fed condition; the 2 beagles of group B took the reference drug orally under fasting condition; the 2 beagles of group C took the test drug orally under fasting condition.

Four hours later after administration, the dogs were allowed to take food or water freely. 5 mL of blood sampling for each dog was done pre-dose and at 0.5, 1, 1.5, 2, 3, 4, 6, 8, 10, 12, 24, 36, and 48 hours after dosing of each period. The samples were centrifuged for 10 minutes at rate of 2500 rotations per minute to separate the plasma. And the plasma was preserved under frozen condition of −20° C. for determination. The adverse reactions of each dog were observed and recorded.

The order of administration was as followed:

TABLE 2
Order of administration

Dog

| Period | 1 | 2 | 3 | 4 | 5 | 6 |
|---|---|---|---|---|---|---|
| 1st period | R | R | T₁ | T₁ | T₂ | T₂ |
| 2^{nd} period | T₁ | T₂ | R | T₂ | R | T₁ |
| 3^{rd} period | T₂ | T₁ | T₂ | R | T₁ | R |

Notes:
R refers to reference drug (tamsulosin hydrochloride sustained-release capsules, Harnal ® Yamanouchi, strength: 0.2 mg per capsule), taken under fasting condition.
T₁ refers to test drug (tamsulosin hydrochloride controlled-release tablets containing 0.2 mg tamsulosin per tablet), taken under fasting conditions.
T₂ refers to test drug (tamsulosin hydrochloride controlled-release tablets containing 0.2 mg tamsulosin per tablet), taken under fed condition.

### Results

### 1. Blood Concentration

The results show that the mean blood concentration of reference drug reaches peak point at about 1 hour after administration. And the mean blood concentrations of test drug under fasting and fed conditions maintain a higher level from 4-8 hours after administration and lower down to base level at 24 hours after administration.

TABLE 3
The mean blood concentration (ng.mL⁻¹) at different times
for test drug compared to reference drug (Mean ± SD)

| Time | Blood concentration (ng · mL⁻¹) | | |
|---|---|---|---|
| (h) | R | T₁ | T₂ |
| 0.5 | 5.28 ± 5.76 | ND ± ND | ND ± ND |
| 1 | 9.48 ± 9.03 | 0.27 ± 0.21 | 0.08 ± ND |
| 1.5 | 9.10 ± 8.71 | 0.63 ± 0.60 | 0.22 ± 0.11 |
| 2 | 6.50 ± 6.18 | 0.84 ± 0.59 | 0.42 ± 0.41 |
| 3 | 4.26 ± 3.01 | 1.04 ± 0.42 | 0.94 ± 0.83 |
| 4 | 1.98 ± 1.25 | 1.63 ± 1.33 | 1.96 ± 1.24 |
| 6 | 0.95 ± 0.61 | 1.87 ± 1.37 | 2.35 ± 1.14 |
| 8 | 0.89 ± 0.96 | 1.98 ± 1.75 | 2.03 ± 1.05 |
| 10 | 0.20 ± 0.14 | 1.28 ± 0.94 | 1.35 ± 0.87 |
| 12 | 0.16 ± 0.07 | 1.75 ± 1.32 | 0.90 ± 1.06 |
| 24 | ND ± ND | 0.37 ± 0.37 | 0.10 ± 0.04 |

Note:
ND - lower than the lowest limit of determination

### 2. Relative Bioavailability

The relative bioavailabilities of test drugs under fasting and fed conditions compared to reference drug are 88.02% and 92.42% respectively. Bioavailability means the amount and rate of unchanged active drug coming into systemic circulation.

TABLE 4
The relative bioavailability for test drugs

| | AUC_{0–24h}(ng · h/mL) | | | BA % | |
|---|---|---|---|---|---|
| | R | T₁ | T₂ | T₁/R | T₂/R |
| Mean ± SD | 28.12 ± 19.97 | 27.21 ± 22.70 | 22.91 ± 11.26 | 88.02 ± 49.72 | 92.42 ± 35.44 |

### 3. Pharmacokinetic Parameters

TABLE 5
The pharmacokinetics parameters for test drug compared to
reference drug (Mean ± SD)

| | AUC_{(0–24)} | AUC_{(0→ ∞)} | Cmax | Tmax | Ke | t_{1/2} | R* |
|---|---|---|---|---|---|---|---|
| | (ng · h/ml) | (ng · h/ml) | (ng/ml) | (h) | (h⁻¹) | (h) | (%) |
| R | 28.13 ± 19.96 | 28.51 ± 20.08 | 10.24 ± 8.79 | 1.25 ± 0.27 | 0.44 ± 0.15 | 1.80 ± 0.88 | 1.26 ± 0.01 |
| T₁ | 27.21 ± 22.70 | 30.24 ± 24.33 | 2.22 ± 1.49 | 5.83 ± 2.40 | 0.18 ± 0.09 | 4.62 ± 2.14 | 10.98 ± 0.11 |
| T₂ | 22.91 ± 11.26 | 23.37 ± 11.36 | 2.72 ± 1.08 | 5.67 ± 1.51 | 0.21 ± 0.05 | 3.51 ± 0.86 | 2.02 ± 0.02 |

*R = [AUC_{(0→ 24)} − AUC_{(0→ ∞)}]/AUC_{(0→ ∞)} × 100%
t_{1/2} The period of time required for the concentration or amount of drug in the blood plasma to be reduced by one-half.
Ke The elimination constant. The fraction of the drug in the body eliminated per unit time is determined by the elimination constant represented by the slope of the line of the log plasma concentration versus time.

According to the results of ANOVA, the Tₘₐₓ of T is remarkably longer than that of R while the Cₘₐₓ of T is remarkably lower than that of R. The results show that the test drug has the feature of controlled release. There is no remarkable difference of bioavailability between fasting and fed conditions for the test drug (P>0.05), indicating that the food effect does not affect the bioavailability of tamsulosin hydrochloride controlled-release tablets.

What is claimed is:

1. An osmotic pump device for administration of the active ingredient tamsulosin or a pharmaceutically acceptable salt thereof at a controlled rate into the gastrointestinal tract comprising:
a) a core comprising a first layer containing a pharmaceutically effective amount of tamsulosin or a pharmaceutically acceptable salt thereof and about 10 to 99 percent by weight of said first layer of polyvinylpyrrolidone polymers and/or copolymers, and a second layer comprising about 10 to 80 percent by weight of said second layer of water-insoluble volume-swellable polymers, about 80 to 10 percent by weight of said second layer of water-soluble osmopolymers, and about 5 to 50 percent by weight of said second layer of osmagents;
b) a wall surrounding said core comprising a semi-permeable material permeable to the passage of an exterior fluid present in said gastrointestinal tract and substantially impermeable to the passing of said tamsulosin or pharmaceutically acceptable salt thereof; and
c) a passageway in said wall communicating with said first layer and the exterior of said device for delivery of said tamsulosin or pharmaceutically acceptable salt thereof from said device.

2. A device according to claim 1 wherein said water-insoluble volume-swellable polymers in said second layer are selected from the group consisting of sodium starch glycolate, low-substituted hydroxypropyl cellulose, crosslinked carboxylmethyl cellulose sodium and mixtures of two or more thereof.

3. A device according to claim 1 wherein said osmopolymers in said second layer are selected from the group consisting of acrylic acid polymers, acrylic acid copolymers, hydroxypropylmethyl cellulose, polyvinylpyrrolidone polymers, polyvinylpyrrolidone copolymer and mixtures of two or more thereof.

4. A device according to claim 1 wherein osmagents in said second layer are selected from the group consisting of a water soluble inorganic salt, mannitol, glucose, lactose, sucrose and mixtures of two or more thereof.

5. A device according to claim 1 wherein said first and second layers each further comprise a lubricant, a glidant, and a colorant.

6. A device according to claim 5 wherein said lubricant and/or glidant is selected from the group consisting of magnesium stearic acid, silicon dioxide and mixtures of two or more thereof.

7. A device according to claim 4 wherein said osmagent in said second layer comprises sodium chloride.

8. A device according to claim 1 wherein said device further comprises plasticizers, light blockers, pore formers, solvents and fillers.

9. A device according to claim 1 wherein said passageway is of the diameter of about 0.2 to 1.2 mm.

10. A device according to claim 1 wherein said wall comprises cellulose acetate or ethyl cellulose.

11. A device according to claim 1 wherein said polyvinylpyrrolidone polymers and/or copolymers in said first layer have an average molecular weight in the range of about 5000 to 3,000,000.
